(19) United States
(12) Patent Application Publication
(10) Pub. No.: US 2008/0219963 A1
(43) Pub. Date: Sep. 11, 2008

(54) Composition Containing Micronutrients With Improved Anti-Oxidant Activity And The Use Thereof

(75) Inventors: Corvi Mora Paolo, Piacenza (IT); Tiziana Canal, Trieste (IT); Federica Ruzzier, Trieste (IT); Fulvio Fortuna, Trieste (IT)

Correspondence Address: CONNOLLY BOVE LODGE & HUTZ LLP, 1875 EYE STREET, N.W., SUITE 1100, WASHINGTON, DC, 20036, US

(73) Assignee: ACTIMEX S.R.L. AREA Science Park, Trieste (IT)

(21) Appl. No.: 11/996,063

(22) PCT Filed: Jul. 18, 2006
(86) PCT No.: PCT/EP2006/064385

§ 371 Date: Jan. 17, 2008

(30) Foreign Application Priority Data
Jul. 19, 2005 (IT) PD2005A000224

(51) Int. Cl. A61K 31/122; A61K 31/047; A61K 36/82; A61K 31/01; A61K 31/385; A61K 31/375

(52) U.S. Cl. 424/94.1; 514/729; 424/729; 514/762; 514/440; 514/691; 514/474

(57) ABSTRACT

Compositions in fine powder form that can be obtained by means of a dry co-grinding process of an at least ternary mixture consisting of an active substance, a carrier and at least one auxiliary co-grinding substance, wherein the active substance is constituted by one or more micronutrient substances with particular antioxidant activity. Such compositions have shown a significant increase in active substance antioxidant power in comparison to solutions of equal active substance content. Said effect is particularly useful for uses for active substances included in antioxidant compositions in the pharmaceutical, cosmetic and dietary-nutritional fields.

### TECHNICAL FIELD

The invention relates to at least ternary compositions that can be obtained through a dry co-grinding process, comprising at least one micronutrient substance particularly with antioxidant activity as active substance, a carrier and at least one co-grinding substance, to a process for the preparation and the pharmaceutical or parapharmaceutical use thereof in the cosmetic and dietary-nutritional fields.

### BACKGROUND ART

Due to their protective biological properties against cellular oxidative stress, for some time now, antioxidant compounds have been believed to be of applicational interest in a number of disorders, for clearly therapeutic purposes, or under paraphysiological conditions, for essentially preventive purposes. Indeed, their applications may be manifold and thus find great use in the pharmaceutical and parapharmaceutical fields, particularly in the cosmetics and dietary-nutritional sectors.

However, many of such compounds are difficult to handle during the industrial preparation processes of compositions suitable for the desired pharmaceutical or parapharmaceutical purposes, since, for example, they are frequently, poorly soluble in both aqueous and organic solvent environments, or possess other unfavourable physico-chemical characteristics.

Besides the above-mentioned drawbacks, and particularly in relation to those pertaining to solubility, from the technological viewpoint, it is essential to also bear in mind that frequently said compounds may not be subjected to overly drastic processes, in order to avoid their degradation due to oxidation phenomena, which would make them practically unusable for the desired purposes.

The formulation related technological difficulties associated with antioxidants are exemplified by the flavonoids, vitamins, mineral salts, polyphenols, lipoic acid, sulphurated aminoacids, EDTA, glutathiones, carotenoids, melatonin, or even a compound such as ubidecarenone. For example, ubidecarenone is a particularly interesting compound due to its biological activities, and for this reason, it has often been used therapeutically in numerous speciality medications with cardiotonic activities. However, it is also known from the pharmaceutical viewpoint, that it is a compound that is very difficult to handle, having a waxy consistency and poor solubility and dispersability. Furthermore, it is characterised by being low-melting, with a melting point of between 45 and 48° C. In relation to solubility, ubidecarenone is not particularly soluble in water or in aqueous environments (<<0.1 mg/ml), while it is poorly soluble in dioxan, ether and methylene chloride. Furthermore, it has very high affinity for plastics.

In order to resolve the problems generally associated with preparing compositions in fine powder form essentially consisting of active substances that are poorly soluble in aqueous or organic environments, this applicant developed a dry co-grinding process in which an active substance is included in a hydrophilic or hydrophobic carrier, depending on the physico-chemical characteristics of the active substance in question, in the presence of an auxiliary co-grinding substance, which allows a significant reduction in co-grinding times, and under milder grinding operational conditions, with undoubted advantages for active substance stability. This process, described in patent application WO03/097012, allows the attainment of active substance/carrier/auxiliary co-grinding substance ternary compositions, the solubility and dissolution characteristics of which are significantly improved with respect to the corresponding active substance/carrier binary compositions.

### SUMMARY

By applying the above described processes to antioxidants in order to obtain compositions in fine powder form that are easily dispersible in aqueous environments, and possibly soluble in the same, the applicant has surprisingly found that the compositions obtained showed a significant increase in antioxidant power considering equal active substance content in solution.

It is therefore an object of the present invention to provide compositions in fine powder form, with good processability, obtainable by means of a dry co-grinding process of an at least ternary composition comprising an active principle, a carrier and at least one auxiliary co-grinding substance, characterised in that said active principle comprises at least one micronutrient substance with antioxidant activity and is present in an amount such that the weight ratio active principle/carrier is less than 1, and said co-grinding process is carried out for less than 90 minutes, whereby the antioxidant activity of said composition is greater than the antioxidant activity of a solution containing the same amount of active substance alone under the same conditions.

According to a preferred aspect of the invention, said carrier is present in an amount not less than 50% w/w of the total of said at least ternary mixture.

According to another preferred aspect of the invention, said enhancement of the antioxidant activity is achieved when the at least ternary composition undergoes a co-grinding process not exceeding 60 minutes.

Further objects of the invention include the process for the preparation thereof and the pharmaceutical or parapharmaceutical use thereof in the cosmetic and dietary-nutritional fields.

### BRIEF DESCRIPTION OF THE FIGURES

FIG. 1: High pressure liquid chromatography (HPLC) of example 6 (lipoic acid/polyvinylpyrrolidone/glycine 1/8.5/0.5) subjected to mechanico-chemical activation for 15 min, and example C (lipoic acid/polyvinylpyrrolidone/glycine 1/8.5/0.5) subjected to mechanico-chemical activation for 90 minutes;

FIG. 2: antioxidant activity of the composition of example 4 (ubidecarenone/γ-cyclodextrin/glycine 1/8/1) subjected to mechanico-chemical activation for 15 min;

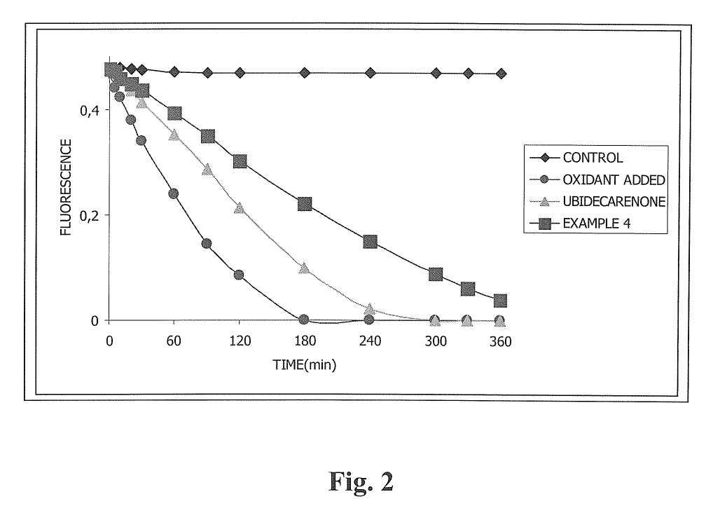

FIG. 3: antioxidant activity of the composition of example 8 (resveratrol/β-cyclodextrin/glycine 1.5/7/1.5) subjected to mechanico-chemical activation for 60 minutes, and example D (resveratrol/β-cyclodextrin/glycine 1.5/7/1.5) subjected to mechanico-chemical activation for 120 minutes.

### DETAILED DESCRIPTION OF THE INVENTION

The objects and advantages of the present invention will be better understood over the course of the following detailed description.

The previously cited patent application WO03/097012 describes a dry co-grinding process wherein an active substance is included in a hydrophilic or hydrophobic carrier, depending on the physico-chemical characteristics of the active substance in question, in the presence of an auxiliary co-grinding substance allowing a significant reduction in co-grinding times. Said process meets the aim of overcoming the technological-pharmaceutical difficulties associated with substances or active principles that are difficult to handle due to their poor solubility and/or stability. Using said process, it is possible to obtain active substance/hydrophilic or hydrophobic carrier/auxiliary co-grinding substance ternary compositions, the solubility and dissolution characteristics of which are significantly improved with respect to the corresponding active substance/carrier binary compositions. Essential for the scope of reducing the co-grinding time and improving the solubility/dispersibility of the active ingredients included in the ternary compositions, is the presence of an auxiliary co-grinding substance selected from the group consisting of aminoacids, malic acid, fumaric acid, ascorbic acid, citric acid, polyalcohols, ethylene diamine tetra acetate, surfactants, lecithins, phospholipids and derivatives thereof, while the hydrophilic carrier may be selected from the group consisting of dextrins and derivatives thereof (including cyclic derivatives), dextrans, linear and cross-linked polyvinylpyrrolidones and derivatives thereof, cellulose and derivatives thereof, mannoglucurans, chitosans, galactomannans and sodium starch glycolate, and the hydrophobic carrier may be selected from the group consisting of ethylcellulose, polyacrylates and derivatives, polymethacrylates and derivatives, polystyrene and derivatives, sylica. For the purposes pursuant to WO03/097012 the auxiliary co-grinding substances were essential, as appears evident from comparison of the ternary compositions with the corresponding binary compositions. For the ternary composition co-grinding process, active substance/carrier/auxiliary co-grinding substance weight ratios of i) active substance/carrier of between 1:0.1 and 1:100 and preferably between 1:0.5 and 1:50; ii) active substance:auxiliary co-grinding substance of between 1:0.1 and 1:20 and preferably between 1:0.2 and 1:10 are envisaged. The co-grinding time was comprised between 0.25 and 24 hours. It has now been found that by subjecting an at least ternary mixture containing antioxidants to mechanico-chemical activation through a co-grinding process, an unexpected enhancement of the antioxidant activity at relatively short grinding times is achieved, besides an improvement in solubility or dispersibility in aqueous environment. Furthermore, surprisingly, such enhancement of the antioxidant activity is not always linked to a greater solubility of the compositions.

The process that brings about the result above is characterised by a particular ratio between the active principle and the carrier. More precisely, the active principle or substance is present in a w/w ratio with the carrier of less than 1, preferably less than 0.8, more preferably less than 0.5.

The carrier is preferably present in a weight percentage of at least 50% on the amount of the at least ternary composition. Preferably the carrier is present in a w/w percentage of at least 60% on the amount of the at least ternary composition.

An enhancement of the antioxidant activity can be obtained when the at least ternary mixture is subjected, through a co-grinding process, to mechanico-chemical activation for a period of time of less than 90 minutes, preferably not greater than 60 minutes. The enhancement of the antioxidant activity of said composition is measured by comparison with the antioxidant activity of the same quantity of active substance alone under the same conditions (in solution).

The dry co-grinding process may be performed using known means, such as ball mills, blade mills, vibrational mills, centrifugal mills and planetary mills.

With the term “dry” it is meant in the present description a process in which no solvents whatsoever are employed and the resulting at least ternary composition has less than 10% by weight of a liquid, preferably less than 5%, more preferably less than 3%.

The preferred active substances or principles generally belong to the class of micronutrients with antioxidant or anti-free radical activity, and may include, but are not limited to ubidecarenone, lipoic acid, lycopene, resveratrol, green tea extracts, astaxantin, pycnogenol, genistein, tocopherols and tocotrienols, retinol, carotenoids, ascorbic acid, glutathione, sulphurated aminoacids and derivatives thereof, flavonoids and mixtures and derivatives thereof, polyphenols and mixtures and derivatives thereof.

For the purposes of the present invention, with the antioxidants, it is preferable to use hydro- or amphiphilic carriers, and particularly carriers selected from the group consisting of dextrins and derivatives thereof (also cyclic), dextrans, linear, branched and cross-linked polyvinylpyrrolidones, cellulose and derivatives thereof, mannoglycosans, chitosans, alginates and derivatives thereof, galactomannans and sodium starch glycolate, as inclusion carriers, while the auxiliary co-grinding substances are selected from the group consisting of aminoacids, weak acids (for example malic acid, fumaric acid, ascorbic acid, citric acid), polyalcohols, ethylene diamine tetra-acetic acid and the salts thereof, surfactants, lecithins, phospholipids and derivatives thereof, and preferably from the group consisting of glycine, lysine, serine and disodium ethylene diamine tetra-acetate.

For the purposes of improving the processability of the ternary mixture consisting of active substance: carrier: auxiliary co-grinding substance, additional (one or more) auxiliary co-grinding substances may be used with various properties and capable of improving, for example, the technological (free flowability, residual humidity) or organoleptic characteristics, such as for example glycyrrhizinate, sorbitol, silicas, chelating agents, aminoacids, sweeteners, inorganic oxides.

Some examples of preparations, on both the laboratory and pilot plant scales, of compositions according to the invention, are described below by way of non-limiting illustration of the present invention.

### EXAMPLE 1

### Ternary Compositions of Ubidecarenone/Copovidone/Glycine 1/8/1

30 g of a 1/8/1 w/w ratio mixture of ubidecarenone (3 g), copovidone (24 g) and glycine (3 g), obtained using a rotating body mixer, were loaded into the jar of a planetary mill and subjected to mechanico-chemical activation for 15 minutes at a speed of 200 rpm. Upon completion of the process, the product, in the form of a fine powder, was unloaded and sieved at 355 μm. A ubidecarenone/copovidone/glycine ternary composite material in a w/w ratio of 1/8/1 was obtained, with a ubidecarenone content of 10%.

### EXAMPLE 2

### Ternary Compositions of Ubidecarenone/β-Cyclodextrin/Glycine 1/8/1

1 kg of 1/8/1 w/w ratio mixture of ubidecarenone (100 g), copovidone (800 g) and glycine (100 g), obtained using a rotating body mixture, was loaded into the chamber of a high energy vibrational mill and subjected to mechanico-chemical activation for 15 minutes. Upon completion of the process, the product, in the form of a fine powder, was unloaded and sieved at 710 μm. A ubidecarenone/copovidone/glycine ternary composite material in a w/w ratio of 1/8/1 was obtained, with a ubidecarenone content of 10%.

### EXAMPLE 3

### Ternary Compositions of Ubidecarenone/Copovidone/Glycine 20/75/5

1 kg of a 20/75/5 w/w ratio mixture of ubidecarenone (200 g), copovidone (750 g) and glycine (50 g), obtained by using a rotating body mixer, was loaded into the chamber of a high energy vibrational mill and subjected to mechanico-chemical activation for 30 minutes. Upon completion of the process, the product, in the form of a fine powder, was sieved at 710 μm. A ubidecarenone/copovidone/glycine ternary composite material was obtained with a weight percentage ratio of 20/75/5.

### EXAMPLE 4

### Ternary Compositions of Ubidecarenone/γ-Cyclodextrin/Glycine 1/8/1

3 g of ubidecarenone, 24 g of γ-cyclodextrin and 3.0 g of glycine were mixed for 10 minutes in a rotating body mixer; the mixture was then loaded into the jar of a planetary mill and subjected to mechanico-chemical activation for 15 minutes at a speed of 200 rpm. Upon completion of the process, the product, in the form of a fine powder, was unloaded and sieved at 355 μm. A ubidecarenone/γ-cyclodextrin/glycine ternary composite material in a w/w ratio of 1/8/1 was obtained with a ubidecarenone content of 10%.

### EXAMPLE 5

### Ternary Compositions of Lipoic Acid/Linear Polyvinylpyrrolidone/Glycine 1/8/1

3 g of lipoic acid, 24 g of linear polyvinylpyrrolidone and 3 g of glycine were mixed for 10 minutes in a rotating body mixer; the mixture was then loaded into the jar of a planetary mill and subjected to mechanico-chemical activation for 15 minutes at a speed of 150 rpm. Upon completion of the process, the product, in the form of a fine powder, was unloaded and sieved at 355 μm. A lipoic acid/linear PVP/glycine ternary composite material with a weight ratio of 1/8/1 was obtained with a lipoic acid content of 10%.

### EXAMPLE 6

### Ternary Compositions of Lipoic Acid/Linear Polyvinylpyrrolidone/Arginine 1/8.5/0.5

3 g of lipoic acid, 25.5 g of linear polyvinylpyrrolidone and 1.5 g of arginine were mixed for 10 minutes in a rotating body mixer; the mixture was then loaded into the jar of a planetary mill and subjected to mechanico-chemical activation for 15 minutes at a speed of 150 rpm. Upon completion of the process, the product, in the form of a fine powder, was unloaded and sieved at 355 μm. A lipoic acid/linear PVP/arginine ternary composite material with a weight ratio of 1/8.5/0.5 was obtained with a lipoic acid content of 10%.

### EXAMPLE 7

### Quaternary Compositions of Resveratrol/β-Cyclodextrin/Glycine/Ammonium Glycyrrhizate 1.5/7.5/0.5/0.5

4.5 g of resveratrol, 22.5 g of beta cyclodextrin 1.5 g of glycine and 1.5 g of ammonium glycyrrhizinate were mixed for 10 minutes in a rotating body mixer, then loaded into the jar of a planetary mill and subjected to mechanico-chemical activation for 30 minutes at a speed of 200 rpm. Upon completion of the process, the product, in the form of a fine powder was unloaded and sieved at 355 μm. A resveratrol/beta-cyclodextrin/glycine/ammonium glycyrrhizinate quaternary composite material with a weight ratio of 1.5/7.5//0.5/0.5 was obtained with a resveratrol content of 15%.

### EXAMPLE 8

### Ternary Compositions of Resveratrol/β-Cyclodextrin/Glycine 1.5/7/1.5

4.5 g of resveratrol, 21.0 g of beta-cyclodextrin and 4.5 g of glycine were mixed for 10 minutes in a rotating body mixer, then loaded into the jar of a planetary mill and subjected to mechanico-chemical activation for 60 minutes at a speed of 200 rpm. Upon completion of the process, the product, in the form of a fine powder was unloaded and sieved at 355 μm. A resveratrol/beta-cyclodextrin/glycine ternary composite material with a weight ratio of 1.5/7/1.5 was obtained with a resveratrol content of 15%.

### EXAMPLE 9

### Ternary Compositions of Lipoic Acid/β-Cyclodextrin/Arginine 2/7/1

6 g of lipoic acid, 21 g of β-cyclodextrin and 3 g of arginine were mixed for 10 minutes in a rotating body mixer; the mixture was then loaded into the jar of a planetary mill and subjected to mechanico-chemical activation for 30 minutes at a speed of 120 rpm. Upon completion of the process, the product, in the form of a fine powder, was unloaded and sieved at 355 μm. A lipoic acid/β-cyclodextrin/arginine ternary composite material with a weight ratio of 2/7/1 was obtained with a lipoic acid content of 20%.

### EXAMPLE 10

### Ternary Compositions of Green Tea Dry Extract/β-Cyclodextrin/Glycine 3.5/5.5/1.0

10.5 g of green tea d.e., 16.5 g of β-cyclodextrin and 3 g of glycine were mixed for 10 minutes in a rotating body mixer; the mixture was then loaded into the jar of a planetary mill and subjected to mechanico-chemical activation for 60 minutes at a speed of 150 rpm. Upon completion of the process, the product, in the form of a fine powder, was unloaded and sieved at 355 μm. A green tea d.e./β-cyclodextrin/glycine ternary composite material with a weight ratio of 3.5/5.5/1.0 was obtained with a green tea d.e. content of 35%.

### EXAMPLE 11

### Ternary Compositions of Green Tea Dry Extract/Povidone/Serine 3.0/6.0/1.0

9 g of green tea d.e., 18 g of povidone and 3 g of serine were mixed for 10 minutes in a rotating body mixer; the mixture was then loaded into the jar of a planetary mill and subjected to mechanico-chemical activation for 30 minutes at a speed of 200 rpm. Upon completion of the process, the product, in the form of a fine powder, was unloaded and sieved at 355 μm. A green tea d.e./povidone/serine ternary composite material with a weight ratio of 3/6/1 was obtained with a green tea d.e. content of 30%.

### EXAMPLE 12

### Quaternary Compositions of Green Tea Dry Extract/β-Cyclodextrin/Serine/Ammonium Glycyrrihizate 4/5/0.5/0.5

12 g of green tea d.e., 15 g of β-cyclodextrin, 1.5 g of serine, 1.5 g of ammonium glycyrrhizate were mixed for 10 minutes in a rotating body mixer; the mixture was then loaded into the jar of a planetary mill and subjected to mechanico-chemical activation for 15 minutes at a speed of 250 rpm. Upon completion of the process, the product, in the form of a fine powder, was unloaded and sieved at 355 μm. A green tea d.e./β-cyclodextrin/serine/ammonium glycyrrhizate quaternary composite material with a weight ratio of 4/5/0.5/0.5 was obtained with a green tea d.e. content of 40%.

### EXAMPLE 13

### Ternary Compositions of Astaxantin/β-Cyclodextrin/Glycine 1.5/8/0.5

4.5 g of astaxantin, 24 g of β-cyclodextrin and 1.5 g of glycine were mixed for 10 minutes in a rotating body mixer; the mixture was then loaded into the jar of a planetary mill and subjected to mechanico-chemical activation for 75 minutes at a speed of 120 rpm. Upon completion of the process, the product, in the form of a fine powder, was unloaded and sieved at 355 μm. A astaxantin/β-cyclodextrin/glycine ternary composite material with a weight ratio of 1.5/8/0.5 was obtained with an astaxantin content of 15%.

### EXAMPLE 14

### Quaternary Compositions of Astaxantin/Povidone/Ascorbic Acid/Zn Gluconate 2.5/5.5/1.5/0.5

7.5 g of astaxantin, 16.55 g of povidone, 4.5 g of ascorbic acid, 1.5 g of zinc gluconate were mixed for 10 minutes in a rotating body mixer; the mixture was then loaded into the jar of a planetary mill and subjected to mechanico-chemical activation for 35 minutes at a speed of 200 rpm. Upon completion of the process, the product, in the form of a fine powder, was unloaded and sieved at 355 μm. A astaxantin/povidone/ascorcic acid/Zn gluconate quaternary composite material with a weight ratio of 2.5/5.5/1.5/0.5 was obtained with an astaxantin content of 25%.

### EXAMPLE 15

### Ternary Compositions of Pycnogenol/β-Cyclodextrin/Glycine 2.5/6.5/1

7.5 g of pycnogenol, 19.5 g of β-cyclodextrin and 3 g of glycine were mixed for 10 minutes in a rotating body mixer; the mixture was then loaded into the jar of a planetary mill and subjected to mechanico-chemical activation for 45 minutes at a speed of 150 rpm. Upon completion of the process, the product, in the form of a fine powder, was unloaded and sieved at 355 μm. A pycnogenol/β-cyclodextrin/glycine ternary composite material with a weight ratio of 2.5/6.5/1 was obtained with a pycnogenol content of 25%.

### EXAMPLE 16

### Quaternary Compositions of Lycopene/Chitosan/Ammonium Glycyrrhizate/Glycine 2/6/1.5/0.5

6 g of lycopene, 18 g of chitosan, 4.5 g of ammonium glycyrrhizate and 1.5 g of glycine were mixed for 10 minutes in a rotating body mixer; the mixture was then loaded into the jar of a planetary mill and subjected to mechanico-chemical activation for 25 minutes at a speed of 200 rpm. Upon completion of the process, the product, in the form of a fine powder, was unloaded and sieved at 355 μm. A lycopene/chitosan/ammonium glycyrrhizate/glycine quaternary composite material with a weight ratio of 2/6/1.5/0.5 was obtained with a lycopene content of 20%.

### EXAMPLE 17

### Quaternary Compositions of Genistein/β-Cyclodextrin/N-Acetylcistein/EDTA 1/7.5/1/0.5

3 g of genistein, 22.5 g of β-cyclodextrin, 3 g of N-acetylcistein and 1.5 g of EDTA were mixed for 10 minutes in a rotating body mixer; the mixture was then loaded into the jar of a planetary mill and subjected to mechanico-chemical activation for 30 minutes at a speed of 200 rpm. Upon completion of the process, the product, in the form of a fine powder, was unloaded and sieved at 355 μm. A genistein/β-cyclodextrin/N-acetylcistein/EDTA quaternary composite material with a weight ratio of 1/7.5/1/0.5 was obtained with a genistein content of 10%.

### EXAMPLE 18

### Ternary Compositions of Genistein/β-Cyclodextrin/Methionine 2/7/1

6 g of genistein, 21 g of β-cyclodextrin, 3 g of methionine were mixed for 10 minutes in a rotating body mixer; the mixture was then loaded into the jar of a planetary mill and subjected to mechanico-chemical activation for 30 minutes at a speed of 200 rpm. Upon completion of the process, the product, in the form of a fine powder, was unloaded and sieved at 355 μm. A genistein/β-cyclodextrin/methionine ternary composite material with a weight ratio of 2/7/1 was obtained with a genistein content of 20%.

### EXAMPLE 19

### Ternary Compositions of Genistein/β-Cyclodextrin/Ascorbic Acid 2/7/1

6 g of genistein, 21 g of β-cyclodextrin, 3 g of ascorbic acid were mixed for 10 minutes in a rotating body mixer; the mixture was then loaded into the jar of a planetary mill and subjected to mechanico-chemical activation for 45 minutes at a speed of 120 rpm. Upon completion of the process, the product, in the form of a fine powder, was unloaded and sieved at 355 μm. A genistein/β-cyclodextrin/ascorbic acid ternary composite material with a weight ratio of 2/7/1 was obtained with a genistein content of 20%.

### EXAMPLE 20

### Ternary Compositions of Genistein/β-Cyclodextrin/Ascorbic Acid 1/4/5

3 g of genistein, 12 g of β-cyclodextrin, 15 g of ascorbic acid were mixed for 10 minutes in a rotating body mixer; the mixture was then loaded into the jar of a planetary mill and subjected to mechanico-chemical activation for 45 minutes at a speed of 120 rpm. Upon completion of the process, the product, in the form of a fine powder, was unloaded and sieved at 355 μm. A genistein/β-cyclodextrin/ascorbic acid ternary composite material with a weight ratio of 1/4/5 was obtained with a genistein content of 10%.

### EXAMPLE 21

### Ternary Compositions of Genistein/β-Cyclodextrin/N-Acetyl Methionine 1/4/5

3 g of genistein, 12 g of β-cyclodextrin, 15 g of N-acetyl methionine were mixed for 10 minutes in a rotating body mixer; the mixture was then loaded into the jar of a planetary mill and subjected to mechanico-chemical activation for 75 minutes at a speed of 120 rpm. Upon completion of the process, the product, in the form of a fine powder, was unloaded and sieved at 355 μm. A genistein/β-cyclodextrin/N-acetyl methionine ternary composite material with a weight ratio of 1/4/5 was obtained with a genistein content of 10%.

### EXAMPLE 22

### Ternary Compositions of Genistein/β-Cyclodextrin/Glutamic Acid 1/5/4

3 g of genistein, 15 g of β-cyclodextrin, 12 g of glutamic acid were mixed for 10 minutes in a rotating body mixer; the mixture was then loaded into the jar of a planetary mill and subjected to mechanico-chemical activation for 60 minutes at a speed of 150 rpm. Upon completion of the process, the product, in the form of a fine powder, was unloaded and sieved at 355 μm. A genistein/β-cyclodextrin/glutamic acid ternary composite material with a weight ratio of 1/5/4 was obtained with a genistein content of 10%.

### EXAMPLE 23

### Ternary Compositions of Ubidecarenone/Copovidone/Glycine 3/6/1

1 kg of a 3/6/1 w/w ratio mixture of ubidecarenone (300 g), copovidone (600 g) and glycine (100 g), obtained using a rotating body mixer, were loaded into a high-energy vibrational mill and subjected to mechanico-chemical activation for 25 minutes. Upon completion of the process, the product, in the form of a fine powder, was unloaded and sieved at 710 μm. A ubidecarenone/copovidone/glycine ternary composite material in a w/w ratio of 3/6/1 was obtained with a ubidecarenone content of 30%.

### EXAMPLE 24

### Ternary Compositions of Ubidecarenone/Copovidone/Glycine 25/65/10

1 kg of a 25/65/10 w/w ratio mixture of ubidecarenone (250 g), copovidone (650 g) and glycine (100 g), obtained using a rotating body mixer, was loaded into a high-energy vibrational mill and subjected to mechanico-chemical activation for 25 minutes. Upon completion of the process, the product, in the form of a fine powder, was unloaded and sieved at 710 μm. A ubidecarenone/copovidone/glycine ternary composite material in a w/w ratio of 25/65/10 was obtained with a ubidecarenone content of 25%.

### COMPARATIVE EXAMPLES

### Example A

### Ternary Compositions of Ubidecarenone/Copovidone/Glycine 1/8/1

30 g of a 1/8/1 w/w ratio mixture of ubidecarenone (3 g), copovidone (24 g) and glycine (3 g), obtained using a rotating body mixer, were loaded into the jar of a planetary mill and subjected to mechanico-chemical activation for 90 minutes at a speed of 200 rpm. Upon completion of the process, a soft unprocessable material was obtained.

### Example B

### Ternary Compositions of Ubidecarenone/Copovidone/Glycine 2/7.510.5

1 kg of a 20/75/5 w/w ratio mixture of ubidecarenone (200 g), copovidone (750 g) and glycine (50 g), obtained using a rotating body mixer, were loaded into the chamber of a high energy vibrational mill and subjected to mechanico-chemical activation for 120 minutes. Upon completion of the process, a soft unprocessable material was obtained.

### Example C

### Ternary Compositions of Lipoic Acid/Linear PVP/Arginine 1/8.5/0.5

3 g of lipoic acid, 25.5 g of linear polyvinylpyrrolidone and 1.5 g of glycine were mixed for 10 minutes in a rotating body mixer, then loaded into the jar of a planetary mill and subjected to mechanico-chemical activation for 90 minutes at a speed of 150 rpm. Upon completion of the process, a soft unprocessable material is obtained.

### Example D

### Ternary Compositions of Resveratrol/β-Cyclodextrin/Glycine 1.5/7/1.5

4.5 g of resveratrol, 21 g of β-cyclodextrin and 4.5 g of glycine were mixed for 10 minutes in a rotating body mixer, then loaded into the jar of a planetary mill and subjected to mechanico-chemical activation for 120 minutes. Upon completion of the process, a sticky unprocessable material was obtained,

### Example E

### Ternary Compositions of Green Tea Dry Extract/Povidone/Serine 3.0/6.0/1.0

9 g of green tea d.e., 18 g of povidone and 3 g of serine were mixed for 10 minutes in a rotating body mixer; the mixture was then loaded into the jar of a planetary mill and subjected to mechanico-chemical activation for 120 minutes. Upon completion of the process, a crusty and not easily processable material was obtained.

### Example F

### Quaternary Compositions of Green Tea Dry Extract/β-Cyclodextrin/Serine/Ammonium Glycyrrhizate 4.5/4.5/0.5/0.5

13.5 g of green tea d.e., 13.5 g of β-cyclodextrin, 1.5 g of serine, 1.5 g of ammonium glycyrrhizate were mixed for 10 minutes in a rotating body mixer; the mixture was then loaded into the jar of a planetary mill and subjected to mechanico-chemical activation for 120 minutes. Upon completion of the process, a product in the form of a powder with poor morphology was unloaded. A green tea d.e./β-cyclodextrin/serine/ammonium glycyrrhizate quaternary composite material with a weight ratio of 4.5/4.5/0.5/0.5 was obtained with a green tea d.e. content of 45%.

### Example G

### Ternary Compositions of Astaxantin/β-Cyclodextrin/Glycine 5.5/4.0/0.5

16.5 g of astaxantin, 12 g of β-cyclodextrin and 1.5 g of glycine were mixed for 10 minutes in a rotating body mixer; the mixture was then loaded into the jar of a planetary mill and subjected to mechanico-chemical activation for 120 minutes. Upon completion of the process, a crust-like product was obtained.

### Example H

### Ternary Compositions of Pycnogenol-Cyclodextrin/Glycine 6.5/2.5/1

19.5 g of pycnogenol, 7.5 g of β-cyclodextrin and 3 g of glycine were mixed for 10 minutes in a rotating body mixer; the mixture was then loaded into the jar of a planetary mill and subjected to mechanico-chemical activation for 45 minutes. Upon completion of the process, a crusty product was unloaded. A pycnogenol/β-cyclodextrin/glycine ternary composite material with a weight ratio of 6.5/2.5/1 is obtained with a pycnogenol content of 65%.

### Example I

### Quaternary Compositions of Lycopene/Chitosan/Ammonium Glycyrrhizate/Glycine 2/6/1.5/0.5

6 g of lycopene, 18 g of chitosan, 4.5 g of ammonium glycyrrhizate and 1.5 g of glycine were mixed for 10 minutes in a rotating body mixer; the mixture was then loaded into the jar of a planetary mill and subjected to mechanico-chemical activation for 120 minutes. Upon completion of the process, a crust-like product was obtained.

### Example L

### Ternary Compositions of Genistein/β-Cyclodextrin/Methionine 2/7/1

6 g of genistein, 21 g of β-cyclodextrin, 3 g of methionine were mixed for 10 minutes in a rotating body mixer; the mixture was then loaded into the jar of a planetary mill and subjected to mechanico-chemical activation for 120 minutes. Upon completion of the process, a sticky unworkable material was obtained.

### Example M

### Ternary Compositions of Genistein/β-Cyclodextrin/Ascorbic Acid 5/4/1

15 g of genistein, 12 g of β-cyclodextrin, 3 g of ascorbic acid were mixed for 10 minutes in a rotating body mixer; the mixture was then loaded into the jar of a planetary mill and subjected to mechanico-chemical activation for 45 minutes. Upon completion of the process, the product, in the form of a fine powder, was unloaded and sieved at 355 μm. A genistein/β-cyclodextrin/ascorbic acid ternary composite material with a weight ratio of 5/4/1 was obtained with a genistein content of 50%.

### Characterisation of the Compositions

The compositions, prepared according to the above-described examples, have been characterised in relation to their residual crystallinity and solubility in buffered water at pH=7, in comparison to the active substance contained therein. In addition, the antioxidant activity has also been evaluated by means of spectrofluormetric measurements in comparison to solutions of equal concentration of the active substance contained therein.

### A. Solubility

An excess of powder is added to buffered water at pH=7 until a precipitate is obtained. The maximum quantity of active substance present in solution is verified after 24 hours (equilibrium) by means of a suitable analytical method (for example UV spectrophotometry, HPLC).

### B. Differential Scanning Calorimetry (DSC)

DSC is a technique which allows evaluation of the crystallinity of powders based on determination of the heat exchanges occurring in the same during melting subsequent to progressive heating.

### C. Antioxidant Activity

Antioxidant activity was measured using the ORAC (Oxygen Radical Absorbance Capacity) test. Said test has been developed by Cao et al. in 1993 (Oxygen-radical absorbance capacity assay for antioxidants. Free Rad. Biol. Med. 1993; 14: 303-11).

The test was based on measuring the inhibition induced by an antioxidant on the loss of activity of a fluorescent indicator, in the presence of oxidants.

In practice, given a fluorescent indicator (fluorescein) which loses its fluorescence due to the action of an oxidising agent (AAPH), antioxidant activity was evaluated in terms of maintenance of the fluorescence of the indicator over time, due to the ability of the antioxidant to counteract the action of the oxidising agent.

Antioxidant activity was assessed by spectrofluorometric assay, measuring the decay of the fluorescence of the indicator over time (from 0 to 360 minutes). A measuring the fluorescence of the sodium fluorescein indicator was performed using a spectrofluorimeter at a wavelength of 515 nm.

Antioxidant activity is expressed as the antioxidant power in relation to Trolox® at the same concentration of the samples.

The characterisation results are reported in the following Tables 1-8.

TABLE 1
UBIDECARENONE

| | | | | | % | | |
|---|---|---|---|---|---|---|---|
| | | Solubility | Increase | Relative | antiox. | Peak | |
| | | (μg/ml) | in | antiox. | activity | T | ΔH |
| PRODUCT | Processability | at pH = 7 | solubility | activity | increase | (° C.) | (J/G) |
| UBIDECARENONE | | 2 | | 1 | | 48 | 163 |
| (alone) | | | | | | | |
| EXAMPLE 1 | GOOD | 80 | 40 | 2.1 | +110% | 43 | 105 |
| COMP. EX. A | VERY POOR | 60 | 30 | 0.8 | −20% | ND | ND |
| | (it melts) | | | | | | |
| EXAMPLE 2 | GOOD | 70 | 35 | 2.1 | +110% | 45 | 115 |
| EXAMPLE 3 | GOOD | 70 | 35 | 2 | +100% | | |
| COMP. EX. B | VERY POOR | 60 | 30 | 1 | 0 | ND | ND |
| | (it melts) | | | | | | |
| EXAMPLE 4 | GOOD | 80 | 40 | 2 | +100% | | |
| EXAMPLE 24 | GOOD | 66 | 33 | 1.5 | +50% | 43 | 110 |
| EXAMPLE 25 | GOOD | 70 | 35 | 1.7 | +70% | | |

TABLE 2
LIPOIC ACID

| | | | | | % | | |
|---|---|---|---|---|---|---|---|
| | | Solubility | Increase | Rel. | antiox. | Peak | |
| | | (μg/ml) | in | antiox. | activity | T | ΔH |
| PRODUCT | Processability | at pH = 7 | solubility | activity | increase | (° C.) | (J/G) |
| LIPOIC ACID | | 30 | | 1 | | 65 | 125 |
| (alone) | | | | | | | |
| EXAMPLE 5 | GOOD | 450 | 15 | 1.5 | +50% | 60 | 7 |
| EXAMPLE 6 | GOOD | 420 | 14 | 1.4 | +40% | 60 | 5 |
| COMP. EX. | VERY POOR (it | DEGRADED | | ND | ND | ND | ND |
| C | melts) | | | | | | |
| EXAMPLE 9 | GOOD | 410 | 14 | 1.3 | +30% | 61 | 9 |

TABLE 3
RESVERATROL

| | | | | | % | | |
|---|---|---|---|---|---|---|---|
| | | Solubility | Increase | Rel. | antiox. | Peak | |
| | | (μg/ml) | in | antiox. | activity | T | ΔH |
| PRODUCT | Processability | at pH = 7 | solubility | activity | increase | (° C.) | (J/G) |
| RESVERATROL | | 150 | | 1 | | 250 | 200 |
| (alone) | | | | | | | |
| EXAMPLE 7 | GOOD | 290 | 1.9 | 1.4 | +40% | 200 | 135 |
| EXAMPLE 8 | GOOD | 340 | 2.3 | 1.7 | +70% | 195 | 114 |
| COMP. EX. D | QUITE STICKY | 300 | 2.0 | 0.8 | −20% | 195 | 90 |

TABLE 4
GREEN TEA (DRY EXTRACT)

| | | Rel. | % antiox. |
|---|---|---|---|
| | | antiox. | Activity |
| PRODUCT | Processability | activity | increase |
| GREEN TEA (dry extract) | | 1 | |
| EXAMPLE 10 | GOOD | 1.8 | +80% |
| EXAMPLE 11 | GOOD | 1.7 | +70% |
| EXAMPLE 12 | GOOD | 1.9 | +90% |
| COMP. EXAMPLE E | CRUSTY | N.D. | N.D. |
| COMP. EXAMPLE F | RATHER POOR | 1 | 0 |

TABLE 5
ASTAXANTIN

| | | Rel. | % antiox. |
|---|---|---|---|
| | | antiox. | Activitiy |
| PRODUCT | Processability | activity | increase |
| ASTAXANTIN (alone) | | 1 | |
| EXAMPLE 13 | GOOD | 1.3 | +30% |
| EXAMPLE 14 | GOOD | 1.6 | +60% |
| COMP. EXAMPLE G | CRUSTY | N.D. | N.D. |

TABLE 6
PYCNOGENOL

| | | Rel. | % antiox. |
|---|---|---|---|
| | | antiox. | Activity |
| PRODUCT | Processability | activity | increase |
| PYCNOGENOL (alone) | | 1 | |
| EXAMPLE 15 | GOOD | 1.3 | +30% |
| COMP. EXAMPLE H | CRUSTY | N.D. | N.D. |

TABLE 7
LYCOPENE

| | | Rel. | % antiox. |
|---|---|---|---|
| | | antiox. | Activity |
| PRODUCT | Processability | activity | increase |
| LYCOPENE (alone) | | 1 | |
| EXAMPLE 16 | GOOD | 1.4 | +40% |
| COMP. EXAMPLE I | CRUSTY | N.D. | N.D. |

TABLE 8
GENISTEIN

| | | | | Rel. | % antiox. |
|---|---|---|---|---|---|
| | | Solubility at | Increase in | antiox. | Activity |
| PRODUCT | Processability | pH 7 μg/ml | solubility | activity | increase |
| GENISTEIN | | 10 | | 1 | |
| (alone) | | | | | |
| EXAMPLE 17 | GOOD | 250 | 25 | 1.6 | +60% |
| EXAMPLE 18 | GOOD | 260 | 26 | 1.5 | +50% |
| EXAMPLE 19 | GOOD | 230 | 23 | 2.1 | +110% |
| EXAMPLE 20 | GOOD | 270 | 27 | 1.7 | +70% |
| EXAMPLE 21 | GOOD | 245 | 24 | 1.8 | +80% |
| EXAMPLE 22 | GOOD | 195 | 19 | 1.6 | +65% |
| COMP. EXAMPLE L | STICKY | N.D. | N.D | 1 | 0 |
| COMP. EXAMPLE M | STICKY | N.D | N.D. | 1 | 0 |

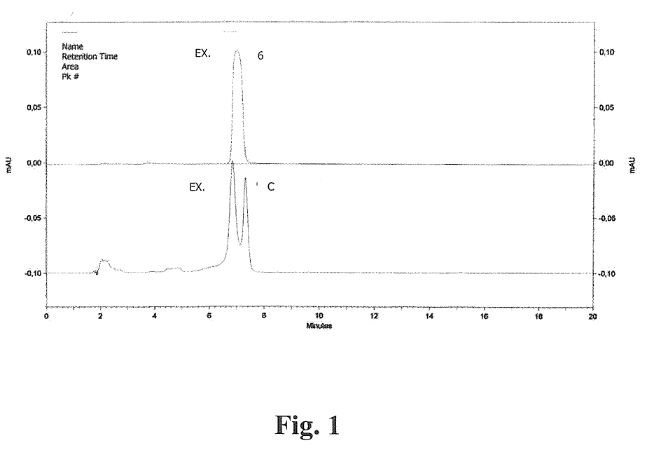

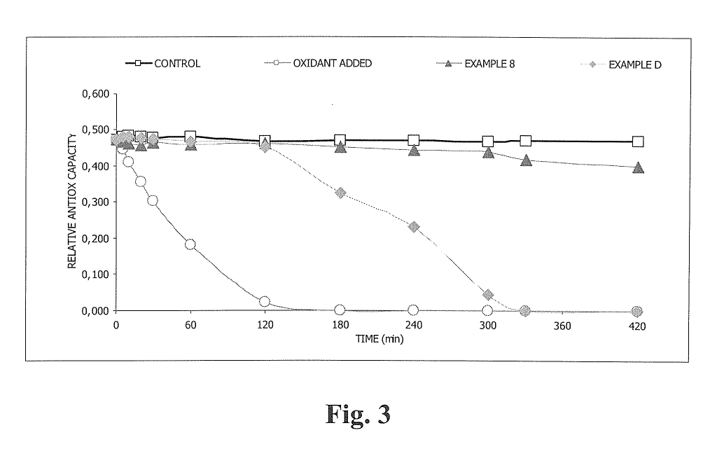

By way of example, FIG. 1 shows comparative chromatograms of example 6 and example C, while FIGS. 3 and 4 show the antioxidant activities of examples 4 and 8, respectively. Analysis of the results obtained shows that the advantageous effect of strengthening of the antioxidant activities of the at least ternary compositions forming the subject of the invention, obtained by means of a co-grinding process, is dependant on the active substance/carrier ratios and on mechanico-chemical activation, which must be less than 90 minutes. Furthermore, it may be observed that said effect is independent of increasing solubility. Furthermore, it may be observed that the addition of the auxiliary co-grinding substance does not have any significant contribution towards said effect.

This indicates that the co-grinding processing parameters, i.e. the defined carrier/active substance w/w ratios and the mechanico-chemical activation times, are the essential conditions for determining the formation of compositions having the characteristics of “multi-composites” which can be exploited due to their increased antioxidant activities in both the pharmaceutical field, for prevention and/or prophylactic therapeutic purposes, and in the parapharmaceutical cosmetic and dietary-nutrition fields. Indeed, the compositions of the invention may be used to prepare products with more favourable active substance quantity/effect ratios. Indeed, the possibility of limiting the quantity of antioxidant with equal antioxidant effect may have a favourable impact on any potential tolerability/toxicity effects. The at least ternary compositions, obtained according to the present invention, in powder form, may thus be made and formulated into products suitable for use for preventive or curative ends as drugs or as dietary supplements or as cosmetics. For such uses, the compositions forming the subject of the present invention may be prepared in powder form, or in mixtures with pharmaceutically, parapharmaceutically, or dietary-nutritional acceptable excipients and diluents. They may additionally be used in various forms, such as for example capsules, tablets, pastes, gels, solutions or suspensions, sprays with pharmaceutically, parapharmaceutically, and dietary-nutritional acceptable excipients and diluents and adapted for such other forms. Furthermore, for parapharmaceutical and cosmetic uses, the compositions of the invention may be formulated with cosmetically acceptable excipients or diluents in the form of lotions, creams, ointments, pastes, gels, patches, mousse, foams, sticks and sprays and other topical forms known for such use.

1. Composition compositions in powder form, with good processability, obtainable by means of a dry co-grinding process of an at least ternary composition comprising an active principle, a carrier and at least one auxiliary co-grinding substance, characterised in that said active principle comprises at least one micronutrient with antioxidant activity and the weight ratio active principle/carrier is less than 1, and said co-grinding process is carried out for less than 90 minutes, whereby the antioxidant activity of said composition is greater than the antioxidant activity of a solution containing the same amount of active substance alone under the same conditions.

2. Composition according to claim 1, characterised in that said carrier is present in an amount not less than 50% w/w of the total of said at least ternary mixture.

3. Composition according to claim 1, characterised in that said co-grinding process is carried out for a time equal to or less than 60 minutes.

4. Composition according to claim 1 wherein said micronutrient with antioxidant activity is selected from the group consisting of ubidecarenone, lipoic acid, lycopene, resveratrol, green tea extracts, astaxantin, pycnogenol, genistein, tocopherols, tocotrienols, retinol, carotenoids, rhodiola, ascorbic acid, glutathiones, sulphurated aminoacids or derivatives thereof, flavonoids or mixtures or derivatives thereof, polyphenols or mixtures or derivatives thereof.

5. Composition according to claim 1 wherein said carrier is a hydrophilic or amphiphilic carrier selected from the group consisting of linear or cyclic dextrins or derivatives thereof, dextrans, linear, branched or cross-linked polyvinylpyrrolidones, cellulose or derivatives thereof, mannoglycosans, chitosans, alginates or derivatives thereof, galactomannans or sodium starch glycolate.

6. Composition according to claim 1 wherein said co-grinding substance is selected from the group consisting of aminoacids, weak acids, polyalcohols, ethylene diamine tetra acetic acid or salts thereof, surfactants, lecithins, phospholipids or derivatives thereof.

7. Composition according to claim 6 wherein the co-grinding substance is selected from the group consisting of glycine, lysine, serine, arginine, methionine, ascorbic acid, glutamic acid and disodium ethylene diamine tetra acetate.

8. Composition according to claim 1 wherein said active principle is present in a w/w ratio with the carrier of less than 0.9.

9. Composition according to claim 8, wherein said active principle is present in a w/w ratio with the carrier of less than 0.5.

10. Composition according to claim 2 wherein said carrier content is comprised between more than 50 up to 99.8% by weight on the amount of the ternary composition.

11. Composition according to claim 1 obtained by a dry co-grinding process in a time comprised between 1 and less than 90 minutes.

12. Composition according to claim 1 wherein one or more other auxiliary co-grinding substances are added to the mixture comprising the active substance, the carrier and at least one auxiliary co-grinding substance.

13. Composition according to claim 12 wherein said other co-grinding substances are selected from the group consisting of ammonium glycyrrhizinate, sorbitol, silicas, chelating agents, aminoacids, sweeteners and inorganic oxides.

14. Use of a composition according to claim 1 for the preparation of a medicament in pharmaceutical forms adapted to administration for therapeutic purposes.

15. Use of a composition according to claim 1 for the preparation of a cosmetic in parapharmaceutical forms that are adapted to dermocosmetic purposes.

16. Use of a composition according to claim 1 for the preparation of a dietary-nutritional product in parapharmaceutical forms that are adapted to dietary supplementation purposes.

17. Dry co-grinding process for obtaining compositions in the form of fine powders according to claim 1.

18. Use of a composition according to any of claims 2 for the preparation of a medicament in pharmaceutical forms adapted to administration for therapeutic purposes.

19. Use of a composition according to any of the claims 2 for the preparation of a cosmetic in parapharmaceutical forms that are adapted to dermocosmetic purposes.

20. Use of a composition according to any of the claims 2 for the preparation of a dietary-nutritional product in parapharmaceutical forms that are adapted to dietary supplementation purposes.
